(19) United States
(12) Patent Application Publication
(10) Pub. No.: US 2024/0165285 A1
(43) Pub. Date: May 23, 2024

(54) DISINFECTION AND ILLUMINATION LAMP AND SYSTEM

(71) Applicants: SHANGHAI SANSI ELECTRONIC ENGINEERING CO. LTD., SHANGHAI (CN); SHANGHAI SANSI TECHNOLOGY CO. LTD., SHANGHAI (CN); JIASHAN SANSI OPTOELECTRONIC TECHNOLOGY CO. LTD., JIASHAN COUNTRY, ZHEJIANG (CN); PUJIANG SANSI OPTOELECTRONIC TECHNOLOGY CO. LTD., JINHUA CITY, ZHEJIANG (CN)

(72) Inventors: BISHOU CHEN, SHANGHAI (CN); MING CHEN, SHANGHAI (CN); SHAN LI, SHANGHAI (CN); XIAOLIANG HE, SHANGHAI (CN)

(73) Assignees: SHANGHAI SANSI ELECTRONIC ENGINEERING CO. LTD., SHANGHAI (CN); SHANGHAI SANSI TECHNOLOGY CO. LTD., SHANGHAI (CN); JIASHAN SANSI OPTOELECTRONIC TECHNOLOGY CO. LTD., JIASHAN COUNTRY, ZHEJIANG (CN); PUJIANG SANSI OPTOELECTRONIC TECHNOLOGY CO. LTD., JINHUA CITY, ZHEJIANG (CN)

(21) Appl. No.: 18/282,540

(22) PCT Filed: May 27, 2021
(86) PCT No.: PCT/CN2021/096206

§ 371 Date: Sep. 18, 2023

(30) Foreign Application Priority Data
Mar. 18, 2021 (CN) 202120559174.7

(51) Int. Cl. A61L 2/10; A61L 2/24; H05B 47/115

(52) U.S. Cl. CPC A61L 2/10; A61L 2/24; H05B 47/115; A61L 2202/11

(57) ABSTRACT

Disclosed are a disinfection and illumination lamp and system. The disinfection and illumination lamp includes: a light source unit (11), including a visible light source (111) and a disinfection light source (112); a sensing unit (12), configured to sense the presence of living beings within a preset range; and a control unit, connected with the light source unit (11) and the sensing unit (12). After the control unit receives a signal transmitted by the sensing unit (12) sensing the presence of the living being, the visible light source (111) is controlled to emit illumination light, and the disinfection light source (112) is turned off; and after the control unit receives a signal transmitted by the sensing unit (12) not sensing the presence of the living being, the visible light source (111) is controlled to emit warning light, and the disinfection light source (112) is controlled to emit disinfection light.

### TECHNICAL FIELD

The present disclosure relates to the technical field of lamps, in particular, to a disinfection and illumination lamp and system.

### BACKGROUND

Disinfection lamps emit ultraviolet light with appropriate wavelengths to destroy genetic materials inside microorganisms, so as to achieve a disinfection effect, and are widely applied in public places such as hospitals, schools, and cinemas, and are of great significance for purifying air, inhibiting the spread of pathogens, and protecting public health. With the increasing awareness of hygiene safety among people, the disinfection lamps are gradually applied and popularized in small private fields such as homes and offices.

However, because the ultraviolet light can kill cells, there is a certain safety risk if the ultraviolet light accidentally irradiates the skin or eyes. In addition, due to the single functionality of the disinfection lamps, the disinfection lamps often remain idle and are low in utilization rate in home or for private use.

### SUMMARY

The present disclosure provides a disinfection and illumination lamp and system, to improve the use safety and utilization rate thereof.

A first aspect of the present disclosure provides a disinfection and illumination lamp, including: a light source unit, including a visible light source and a disinfection light source; a sensing unit, configured to sense the presence of living beings within a preset range; and a control unit, connected with the light source unit and the sensing unit. After the control unit receives a signal transmitted by the sensing unit sensing the presence of the living being, the visible light source is controlled to emit illumination light, and the disinfection light source is turned off; and after the control unit receives a signal transmitted by the sensing unit not sensing the presence of the living being, the visible light source is controlled to emit warning light, and the disinfection light source is controlled to emit disinfection light.

In a preferred implementation of the first aspect of the present disclosure, the disinfection and illumination lamp further includes an aluminum radiator; and the light source unit includes a ceramic substrate, and the ceramic substrate is attached to the aluminum radiator for combined heat dissipation.

In a preferred implementation of the first aspect of the present disclosure, the sensing unit and the light source unit are of a nested structure, and the sensing unit is nested in an inner side of the light source unit.

In a preferred implementation of the first aspect of the present disclosure, the disinfection and illumination lamp further includes a lampshade, and the lampshade includes: a sensing cover, covering the sensing unit; a light source cover, covering the light source unit, and provided with a first through hole for the sensing cover to be embedded; and an outer cover, fixing the light source cover to a fixed position, and provided with a second through hole for the light source cover to be embedded. Most of sensing signals transmitted by the sensing unit enter a sensing area after passing through the sensing cover, the first through hole, and the second through hole. Most of light emitted by the light source unit radiates outwards after passing through the light source cover and the second through hole.

In a preferred implementation of the first aspect of the present disclosure, the sensing unit is nested in the inner side of the light source unit through a printed circuit board (PCB) adapter board, and the sensing cover and the PCB adapter board are detachably connected.

In a preferred implementation of the first aspect of the present disclosure, the light source cover includes a glass cover, and the glass cover seals the light source unit through a sealing ring.

In a preferred implementation of the first aspect of the present disclosure, the ceramic substrate is fixed to the aluminum radiator through thermal conduction adhesive paste.

In a preferred implementation of the first aspect of the present disclosure, the disinfection and illumination lamp further includes: a snap-fit power supply and a lamp housing, and the power supply is snap-fitted with an inner wall of the lamp housing.

In a preferred implementation of the first aspect of the present disclosure, the visible light source and the disinfection light source are of a nested structure, and the visible light source is evenly arranged on a periphery of the disinfection light source.

In order to achieve the above objectives and other related objectives, a second aspect of the present disclosure provides a disinfection and illumination system, including at least one aforementioned disinfection and illumination lamp and a remote control terminal in communication connection with the disinfection and illumination lamp. The remote control terminal is used for remotely controlling the disinfection and illumination lamp.

In summary, the disinfection and illumination lamp and system in the present disclosure have the following beneficial effects: by combining the visible light source and the disinfection light source on a same lamp, multiple functions of the lamp are integrated, which is beneficial to increase the use rate, that is, the illumination function can be realized when disinfection is not required; the sensing unit senses the presence of the living beings within the preset range; after the presence of the living beings in the sensing area are sensed, the disinfection light source is controlled to be turned off so as to prevent the living beings from being exposed to the disinfection light and harmed, and the visible light source can provide illumination for the living beings in the area at the same time; when no living beings are sensed in the sensing area, the disinfection light source emits the disinfection light for disinfection, and the visible light source emits warning light to alert the living beings outside the sensing area, thereby preventing unnecessary harm due to accidental entry; by utilizing the ceramic substrate and the aluminum radiator for heat dissipation together, the heat dissipation efficiency of the lamp is greatly improved; due to the nested structure of the sensing unit and the light source unit, the integration degree between the sensing area and the illumination area is further improved; and a combined nested structure of the sensing cover, the light source cover, and the outer cover designed in correspondence with the sensing unit and the light source unit is stable, and also reduces attenuation of the sensing signals and disinfection and illumination light.

### BRIEF DESCRIPTION OF THE DRAWINGS

FIG. 1 illustrates a schematic exploded view of a disinfection and illumination lamp according to an embodiment of the present disclosure;

FIG. 2 illustrates a section view of a disinfection and illumination lamp according to an embodiment of the present disclosure; and

FIG. 3 illustrates a schematic structural diagram showing part of a disinfection and illumination lamp according to an embodiment of the present disclosure.

### DETAILED DESCRIPTION

Implementations of the present disclosure are described by following specified specific embodiments, and those skilled in the art will easily understand other advantages and effects of the present disclosure disclosed in this specification.

It is to be noted that structures, proportions, sizes, etc. illustrated in the accompanying drawings of this specification are merely used for assisting those skilled in the art in understanding and reading the disclosed content of this specification, but are not intended to limit the implementable limiting conditions of the present disclosure, and thus, do not have substantial significance. Any modifications to the structures, changes in proportional relation, or adjustments in size still fall within the scope covered by the disclosed technical content of the present disclosure without affecting the effects and objectives achieved by the present disclosure. The following detailed descriptions are not regarded as limiting, and the scope of the embodiments of the present disclosure is only limited by the claims of the published patent. The terms used herein are merely used for describing the specific embodiments rather than limiting the present disclosure. Spatial terms such as “top”, “bottom”, “left”, “right”, “below”, “under”, “lower”, “above”, “upper”, etc. can be used in this specification to facilitate the description of the relationship between a component or feature and another component or feature shown in the figures.

In the present disclosure, unless otherwise explicitly specified and limited, the terms “install”, “link”, “connect”, “fix” and “retain” are to be understood in a broad sense. For example, a connection may be a fixed connection, a detachable connection, or an integrated connection; or the connection may be a mechanical connection or an electrical connection; or the connection may be a direct connection, an indirect connection through an intermediary, or internal communication between two components. Those of ordinary skill in the art may understand the specific meanings of the foregoing terms in the present disclosure according to specific situations.

Moreover, as used in this specification, the singular forms “one” “a/an” and “the” are intended to also include plural forms unless the context indicates otherwise. It is to be further understood that the terms “comprise” and “include” indicate the presence of the said features, operations, components, modules, items, types, and/or groups, without excluding the presence, occurrence, or addition of one or more other features, operations, components, modules, items, types, and/or groups. The terms “or” and “and/or” used here are interpreted as inclusive, or mean any one or any combination. Thus, “A, B, or C” or “A, B, and/or C” means “any one of the following: A; B; C; A and B; A and C; B and C; and A, B, and C”. The defined exception occurs only when the combination of components, functions, or operations inherently and mutually exclude each other in certain ways.

The present disclosure provides a disinfection and illumination lamp and system. The use safety and utilization rate of a disinfection lamp are increased by improving its functions and structure.

In order to provide a clearer understanding of the objectives, technical solutions, and advantages of the present disclosure, the technical solutions according to the embodiments of the present disclosure are further described in detail by combining the following embodiments and the accompanying drawings. It is to be understood that the specific embodiments described herein are only used for explaining the present disclosure, but are not intended to limit the present disclosure.

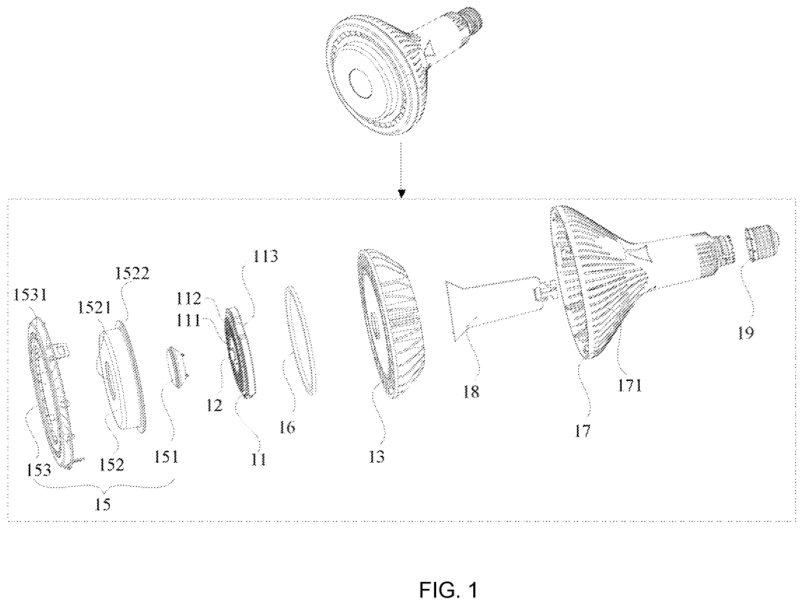

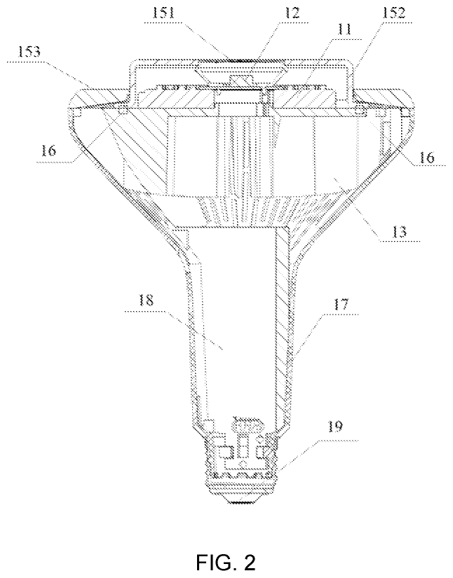

As shown in FIG. 1, an embodiment of the present disclosure provides a schematic exploded view of a disinfection and illumination lamp. FIG. 2 is a section view of the disinfection and illumination lamp. The disinfection and illumination lamp includes: a light source unit 11, a sensing unit 12, and a control unit (not shown in the figure, and may be arranged inside the sensing unit 12). The light source unit 11 includes a visible light source 111 and a disinfection light source 112. Moreover, the control unit is connected with the light source unit 11 and the sensing unit 12. The sensing unit 12 can sense the presence of living beings (e.g., humans and animals) within a preset range. When the control unit receives a signal transmitted by the sensing unit 12 sensing the presence of the living being, the control unit controls the visible light source 111 to emit illumination light and makes the disinfection light source 112 turned off. When the control unit receives a signal transmitted by the sensing unit 12 not sensing the presence of the living being, the control unit controls the visible light source 111 to emit warning light and makes the disinfection light source 112 emit disinfection light. Various units may be expressed as below.

Specifically, the light source unit 11 may be an electroluminescent light source (e.g., a field-luminescent light source and a light-emitting diode (LED) chip), a thermal radiation light source (e.g., an incandescent lamp and a halogen tungsten lamp), or a gas discharge light source (e.g., a xenon lamp, a sodium lamp, and a mercury lamp). In this embodiment, the light source unit 11 is preferably an LED chip, which has the outer dimension flexibly designed, is particularly suitable for the design requirements of combining the visible light source and the disinfection light source in the present disclosure, and is also environmentally-friendly and energy-saving.

The visible light source 111 can emit both the illumination light and the warning light. The warning light may achieve a warning function through flickering at a certain frequency, and may also perform warning about ongoing disinfection work by emitting light such as red light and purple light, indicating that the area cannot be approached. The warning function of the visible light source in the present disclosure may also be achieved by connecting with other alarm circuits, such as one or more of a whistle alarm, a voice alarm, a text alarm, and a short message alarm, which is not limited in this embodiment.

The disinfection light source 112, namely, an ultraviolet light source, may include one or more of a long-wave ultraviolet A (UVA) light source, a medium-wave ultraviolet B (UVB) light source, a short-wave ultraviolet C (UVC) light source, and a vacuum ultraviolet (VUV) light source, etc., for disinfection. The disinfection light source is preferably a UVC LED chip, which has the advantages of high disinfection efficiency, wide disinfection range, safety and environmental protection, etc., such that the disinfection and illumination lamp can rapidly kill more pathogenic bacteria in short time, disinfection work time is shortened, and the use safety of the disinfection and illumination lamp is improved.

The sensing unit 12 may include one or more of an infrared sensor, an ultrasonic sensor, an audio sensor, and a radar sensor, etc. The sensing unit 12 is preferably an infrared sensor in this embodiment, and the infrared sensor includes an infrared living being sensor and an infrared range sensor. By using the infrared living being sensor, information on whether the living being exists or not is acquired based on the difference in infrared radiation characteristics between the living being and a non-living thing, and meanwhile, the infrared range sensor is utilized for acquiring location information of the living being, thereby transmitting a signal to the control unit sensing the presence or absence of the living being within the preset range. In this preferred implementation, living being information and distance information are respectively detected, and compared with the manner of judging whether the living being exists or not and then giving an alarm based on whether infrared rays within a certain space are interrupted or not, this preferred implementation has higher accuracy and anti-interference performance, thereby further improving the use safety of the disinfection and illumination lamp.

The control unit is connected with the light source unit 11 the sensing unit 12. The control unit may include: a microcontroller unit (MCU), a general-purpose processor such as a central processing unit (CPU) and a network processor (NP), a digital signal processor (DSP), an application specific integrated circuit (ASIC), a field-programmable gate array (FPGA), or another programmable logic device, discrete gate or transistor logic device, discrete hardware component, etc.

In some examples, switchover of work modes of the disinfection and illumination lamp may also be achieved by an external controller. The external controller is in communication connection with the light source unit 11 and the sensing unit 12 in a lamp, and is arranged outside the disinfection and illumination lamp. For example, a user may control the disinfection and illumination lamp by devices such as a mobile phone app, a remote control, and a remote terminal. Specifically, after the external controller receives the signal transmitted by the sensing unit 12 sensing the presence of the living being, the visible light source 111 is controlled to emit illumination light, and the disinfection light source 112 is turned off; and after the external controller receives the signal transmitted by the sensing unit 12 not sensing the presence of the living being, the visible light source 111 is controlled to emit warning light, and the disinfection light source 112 is controlled to emit disinfection light.

It is to be noted that a hardware apparatus provided in the present disclosure does not involve any software technology updates regardless of analysis and processing of information collected by the sensing unit, or regulation and control on the light source unit by the control unit. The present disclosure may be used independently or in conjunction with existing programs or software. However, the present disclosure does not involve any software technology updates.

In a preferred implementation of this embodiment, the disinfection and illumination lamp further includes an aluminum radiator 13. The light source unit 11 includes a ceramic substrate 113, and the ceramic substrate 113 is attached to the aluminum radiator 13 for combined heat dissipation. This implementation combines the advantages of the ceramic substrate and the aluminum radiator, which ensures the heat dissipation efficiency of the product while improving the carrying capacity of the product. In some examples, the ceramic substrate 113 is fixed to the aluminum radiator 13 through thermal conduction adhesive paste, providing bonding and sealing functions, as well as having excellent heat dissipation performance.

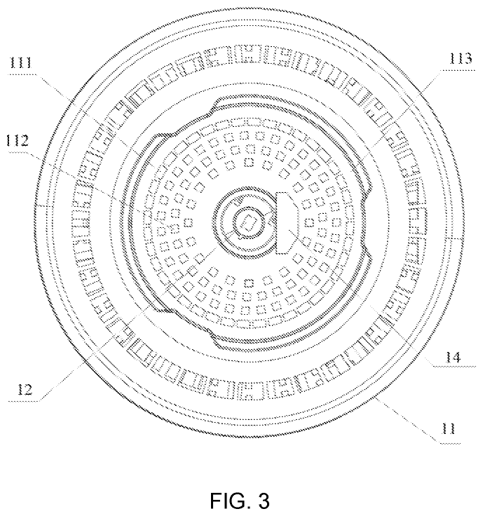

FIG. 3 illustrates a schematic structural diagram showing part of a disinfection and illumination lamp. The light source unit 11 and the sensing unit 12 are of a nested structure, and the sensing unit 12 is nested in an inner side of the light source unit 11. In some examples, the sensing unit 12 may be connected with the light source unit 11 through a printed circuit board (PCB) adapter board 14. The control unit in the sensing unit 12 is connected with the light source unit 11 through the PCB adapter board 14 to control work modes of the light source unit 11, thereby facilitating respective replacement and assembly of components and replacement of local fault components, reducing the after cost of the product, adjusting the sensing range of the sensing unit according to application places, and improving applicability of the product.

In a preferred implementation of this embodiment, the disinfection and illumination lamp includes a lampshade 15. The lampshade 15 further includes a sensing cover 151, a light source cover 152, and an outer cover 153. The sensing cover 151 covers the sensing unit 12. The light source cover 152 covers the light source unit 11, and is provided with a first through hole 1521 for the sensing cover 151 to be embedded. The outer cover 153 fixes the light source cover 152 to a fixed position, and is provided with a second through hole 1531 for the light source cover 152 to be embedded. Moreover, most of sensing signals transmitted by the sensing unit 12 enter a sensing area after passing through the sensing cover 151, the first through hole 1521, and the second through hole 1531. Most of light emitted by the light source unit 11 radiates outwards after passing through the light source cover 152 and the second through hole 1531. In this implementation, the lampshade matched with the nested structure of the light source unit 11 and the sensing unit 12 is designed, such that internal components of the lamp can be prevented from being influenced by environmental factors such as outside dust and moisture, and in addition, the sensing cover and the light source cover matched with the lampshade can be designed according to characteristics of the sensing signals and the light of the light source, thereby reducing signal attenuation, improving accuracy of the sensing unit and the illumination effect, the warning effect, and the disinfection effect of the light source.

In a preferred implementation of this embodiment, the visible light source 111 and the disinfection light source 112 are of the nested structure, and the visible light source 111 is evenly arranged on a periphery of the disinfection light source 112. The visible light source 111 is arranged on the periphery of the disinfection light source 112, which can better realize the warning function, make the disinfection range smaller than the warning range, and allow individuals to promptly notice warning information before entering the disinfection area, thereby avoiding disinfection interrupting, and ensuring the normal disinfection work.

In a preferred implementation of this embodiment, the sensing unit 12 is nested in the inner side of the light source unit 11 through the PCB adapter board 14, and the sensing cover 151 and the PCB adapter board 14 are detachably connected by bonding, snap fit, threaded connection, etc. Snap fit is preferably selected in this embodiment, which facilitates disassembly and assembly of the product.

In a preferred implementation of this embodiment, a glass cover is selected as the light source cover 152, and the glass cover seals the light source unit 11 through a sealing ring 16. The glass cover 152 has the characteristics of excellent and uniform light transmission and high temperature resistance, and can be used for a long time without discoloration or odor, which is beneficial to improve the disinfection and illumination effect of the present disclosure, and meets the demand for fresh air in some disinfection application environments; and the glass cover has a better sealing effect, which is beneficial to protect the internal components of the lamp, improves the use performance of the lamp, and prolongs the service life of the lamp.

In a preferred implementation of this embodiment, the outer cover 153 clamps the glass cover 152 to a clamp position. Specifically, a bottom of the glass cover 152 extends outwards to form a clamping portion 1522. The outer cover 153 is pressed along the clamping portion 1522 and connected to a lamp housing 17 of the lamp, thereby clamping and fixing the glass cover. The outer cover 153 and the lamp housing 17 are detachably connected, preferably through snap fit.

In a preferred implementation of this embodiment, the disinfection and illumination lamp further includes: a snap-fit power supply 18. The snap-fit power supply 18 is snap-fitted with an inner wall of the lamp housing 17. The snap-fit power supply 18 may include a lithium battery, a dry battery, and a solar battery, etc., which is not specifically limited in this embodiment.

In a preferred implementation of this embodiment, the aluminum radiator 13 is of a cooling fin structure, preferably a structure having multiple cooling fins arranged in a loop. The lamp housing 17 is provided with slots 171 matched with the multiple cooling fins of the aluminum radiator 13. Thus, the lamp housing 17 can clamp, connect with, and fix the aluminum radiator 13, and meanwhile further improve the heat dissipation efficiency of the disinfection and illumination lamp.

In a preferred implementation of this embodiment, the disinfection and illumination lamp further includes: a lamp holder 19 used for fixing the disinfection and illumination lamp to an external installation position. The lamp holder in FIG. 1 is a commonly used threaded lamp holder. However, a movable lamp holder may also be selected in the present disclosure. The product is installed at the installation position in a movable manner, such as a pulley manner and a rotation manner, which can implement movable disinfection in the working process, benefit the expansion of the disinfection area, and improve the disinfection effect of the product.

In some embodiments, the lamp holder 19 may be connected with an external power supply to directly supply power to an electrical unit of the disinfection and illumination lamp, or to charge the snap-fit power supply 18, thereby avoiding insufficient electrical energy that may affect the use safety or disinfection and illumination effect of the disinfection and illumination lamp.

To facilitate understanding of the present disclosure, application scenarios are illustrated as below: for example, the disinfection and illumination lamp may be applied in a frozen storage place to disinfect and deodorize the place. When the disinfection lamp is turned on and works, a worker can obtain information about ongoing disinfection operation from a distance through the warning light (flickering or special colored light), thereby preventing the worker from entering accidentally. For another example, the disinfection and illumination lamp may be applied in the household sector to disinfect home appliances, kitchen utensils, or other items. When the disinfection and illumination lamp is turned on and works, if children and pets accidentally enter, the sensing unit 12 can sense the presence of the living beings within the preset range to make the disinfection light source 112 promptly controlled and turned off, thereby ensuring the use safety.

Another embodiment of the present disclosure provides a disinfection and illumination system, including the aforementioned disinfection and illumination lamp and a remote control terminal. The remote control terminal is in communication connection with the disinfection and illumination lamp to remotely control the disinfection and illumination lamp. Specifically, the remote control terminal may be in wired communication, or wireless communication with the disinfection and illumination lamp. A wireless communication circuit includes but not limited to: one or more of a 3G/4G/5G communication circuit, an NB-IoT communication circuit, a LoRa communication circuit, a Zigbee communication circuit, a Z-wave communication circuit, a Wifi communication circuit, a bluetooth communication circuit, an infrared communication circuit, etc.

Optionally, the remote control terminal may acquire signals indicating the presence of living beings in the sensing area through direct communication connection with the sensing unit, thereby controlling the disinfection and illumination lamp to carry out disinfection and warning, or illumination; and the remote control terminal may also be connected with the control unit to acquire control information, thereby performing remote monitoring on the working state of the disinfection and illumination lamp.

In summary, by combining the visible light source and the disinfection light source on a same lamp, multiple functions of the lamp are integrated, which is beneficial to increase the use rate, that is, the illumination function can be realized when disinfection is not required; the sensing unit senses the presence of the living beings within the preset range; after the presence of the living beings in the sensing area are sensed, the disinfection light source is controlled to be turned off so as to prevent the living beings from being exposed to the disinfection light and harmed, and the visible light source can provide illumination for the living beings in the area at the same time; when no living beings are sensed in the sensing area, the disinfection light source emits the disinfection light for disinfection, and the visible light source emits warning light to alert the living beings outside the sensing area, thereby preventing unnecessary harm due to accidental entry; by utilizing the ceramic substrate and the aluminum radiator for heat dissipation together, the heat dissipation efficiency of the lamp is greatly improved; due to the nested structure of the sensing unit and the light source unit, the integration degree between the sensing area and the illumination area is further improved; and a combined nested structure of the sensing cover, the light source cover, and the outer cover designed in correspondence with the sensing unit and the light source unit is stable, and also reduces attenuation of the sensing signals and disinfection and illumination light. Thus, the present disclosure effectively overcomes various defects in the prior art, and has a high industrial utility value.

The above embodiments merely describe the principle and effects of the present disclosure by way of examples, and are not intended to limit the present disclosure. Any person familiar with this technology can modify or change the above embodiments without departing from the spirit and scope of the present disclosure. Thus, any equivalent modifications or changes made by those skilled in the art without departing from the spirit and technical concept disclosed by the present disclosure still fall within the scope of the claims of the present disclosure.

1. A disinfection and illumination lamp, comprising:
a light source unit, comprising a visible light source and a disinfection light source;
a sensing unit, configured to sense presence of a living being within a preset range; and
a control unit, respectively connected with the light source unit and the sensing unit, wherein the control unit is configured such that after the control unit receives a signal transmitted by the sensing unit sensing the presence of the living being, the visible light source is controlled to emit illumination light, and the disinfection light source is turned off; and after the control unit receives a signal transmitted by the sensing unit not sensing the presence of the living being, the visible light source is controlled to emit warning light, and the disinfection light source is controlled to emit disinfection light.

2. The disinfection and illumination lamp according to claim 1, further comprising an aluminum radiator; and the light source unit comprising a ceramic substrate, and the ceramic substrate being attached to the aluminum radiator for combined heat dissipation.

3. The disinfection and illumination lamp according to claim 1, wherein the sensing unit and the light source unit are of a nested structure, wherein the sensing unit is nested in an inner side of the light source unit.

4. The disinfection and illumination lamp according to claim 3, comprising a lampshade, and the lampshade comprising:
a sensing cover, covering the sensing unit;
a light source cover, covering the light source unit, and provided with a first through hole for the sensing cover to be embedded; and
an outer cover, fixing the light source cover to a fixed position, and provided with a second through hole for the light source cover to be embedded,
wherein most of sensing signals transmitted by the sensing unit enter a sensing area after passing through the sensing cover, the first through hole, and the second through hole, and most of light emitted by the light source unit radiates outwards after passing through the light source cover and the second through hole.

5. The disinfection and illumination lamp according to claim 4, wherein the sensing unit is nested in the inner side of the light source unit through a printed circuit board (PCB) adapter board, and the sensing cover and the PCB adapter board are detachably connected.

6. The disinfection and illumination lamp according to claim 4, wherein the light source cover comprises a glass cover, and the glass cover seals the light source unit through a sealing ring.

7. The disinfection and illumination lamp according to claim 2, wherein the ceramic substrate is fixed to the aluminum radiator through thermal conduction adhesive paste.

8. The disinfection and illumination lamp according to claim 1, comprising: a snap-fit power supply and a lamp housing, and the power supply being snap-fitted with an inner wall of the lamp housing.

9. The disinfection and illumination lamp according to claim 1, wherein the visible light source and the disinfection light source are of a nested structure, and the visible light source is evenly arranged on a periphery of the disinfection light source.

10. A disinfection and illumination system, comprising:
the disinfection and illumination lamp according to claim 1; and
a remote control terminal, in communication connection with the disinfection and illumination lamp to remotely control the disinfection and illumination lamp.
